(19) United States
(12) Patent Application Publication
(10) Pub. No.: US 2009/0214576 A1
(43) Pub. Date: Aug. 27, 2009

(54) TYROSINE KINASE INHIBITOR COMPOSITIONS AND METHODS FOR MANUFACTURING AND USING THEM IN THE TREATMENT OF DISEASE

(75) Inventors: Sarah Bacus, Hinsdale, IL (US); Jason Hill, Chicago, IL (US)

Correspondence Address: K&L Gates LLP, P.O. Box 1135, CHICAGO, IL, 60690, US

(73) Assignee: TARGETED MOLECULAR DIAGNOSTICS, LLC, Westmont, IL (US)

(21) Appl. No.: 11/817,956

(22) PCT Filed: Mar. 6, 2006
(86) PCT No.: PCT/US06/07984

§ 371 Date: Feb. 2, 2009

Related U.S. Application Data
(60) Provisional application No. 60/659,263, filed on Mar. 7, 2005.

(51) Int. Cl. C07K 19/00; C12N 5/06; A61K 38/16; A61P 35/00

(52) U.S. Cl. 424/178.1; 435/375; 514/12

(57) ABSTRACT

The invention is the method of using the soluble modified receptor erbB3 to treat breast cancer by competitively inhibiting ligands, such as heregulin, from binding to erbB3 tyrosine kinase receptors on cell surfaces.

### FIELD OF INVENTION

The present invention relates generally to the field of receptor tyrosine kinases and their activation ligands, the use of genetic engineering to create soluble ErbB ligand binding molecules, to the insertion of the modified construct into recombinant DNA vectors to produce a modified form of the receptor that is secreted in soluble form by cells and to the use of such molecules in the treatment of disease.

### BACKGROUND OF THE INVENTION

Cancer is the general name for a group of diseases that together, are a leading cause of death in many countries. Simply put, cancers are diseases that are due to the abnormal proliferation of damaged, out-of-control cells. The abnormal cell growth occurs because of a mutation in some critical gene or group of genes that control normal cell growth, development and death. As these abnormal cells grow, tumors form. In the worst case, the tumors become large enough or prevalent enough throughout the body to produce many adverse effects on the body and ultimately to death.

A primary method of treating cancers is by surgery in an attempt to remove the tumor, thereby stopping its invasion of healthy tissue. Surgery can be a high risk endeavor, and may not be appropriate for every patient. It is also not possible to use surgery to combat every tumor, such as when tumors are found in areas of the brain that cannot be operated on, or in conditions such as leukemias.

As a result, alternative therapies have been and are being designed to treat tumors where surgery is not warranted or possible. One such alternative is radiation. Irradiating tumors causes death or damage to the targeted tumor cells and to collaterally exposed cells that may not be cancer cells. Debilitating side effects are well known.

Chemotherapy is another attempt to destroy tumor tissue in the body by giving cytotoxic compounds to the patient. This type of therapy may be used on its own, as the sole method of fighting the cancer, or either before or after surgery or radiation therapy. As in radiation, the debilitating side effects are well know, and may be of such an extent that it is more dangerous to the well being of the patient than either surgery or radiation.

In attempts to avoid the damage caused to noncancerous tissue, and to avoid the side effects of these various types of therapies, therapies directed solely to obliterating cancerous tissue have been investigated. One such therapy is the use of antibodies specifically directed to tumors, such as colon tumors, thereby attempting to spare healthy tissue. The success rate of such therapy is low.

Genetic based therapies are also being investigated. It has been shown that some gene mutations produce a protein (or target) that responds differently to a drug than does the normal form of the protein that is encoded by the wild-type (or normal) gene. One example of this type of treatment is that done using the drug Gleevec® (Gleevec® is the trade name of imatinib), a highly effective treatment for chronic myelogenous leukemia (“CML”), where it inhibits the oncogene that primarily drives CML progression, known as BCR-ABL kinase. In addition, Gleevec® was found to be an inhibitor of certain tyrosine kinase receptors, especially the mutated forms of the c-kit oncogenic receptor found in many gastrointestinal stomach tumors (“GIST”).

Another similar therapeutic agent, gefitinib (Iressa®, a trademark of AstraZeneca) is a small molecule inhibitor that targets the tyrosine kinase activity of epidermal growth factor receptors (“EGFR”). Iressa® was approved by the FDA for treatment of nonsmall cell lung cancer in patients whose tumors failed to respond to platinum-based and docetaxel chemotherapies. Although only approximately 10% of patients have responded to Iressa®, this subpopulation did show a good clinical response to the drug.

Another approach is to attack the receptors that control the formation of new blood vessels in tumors, or angiogenesis, and to inhibit it. It is believed that a supply of blood vessels to nourish the growing tumor is crucial for its growth. Vascular endothelial growth factor (“VEGF”) has been found to be crucial in this process. The VEGF pathway begins when VEGF binds to its receptors on endothelial cells. These receptors, VEGFR1 and VEGFR2, are transmembrane tyrosine kinases that bind to VEGF on their extracellular domains, which activates the intrinsic tyrosine kinase activity, and initiates intracellular signaling. Blocking of this pathway has been reported using blocking anti-VEGF antibodies or anti-VEGFR antibodies, soluble receptors that prevent VEGF from binding to its receptors by acting as traps, and small molecule inhibitors of the tyrosine kinase activity of the VEGFR's. Holash, et al. have reported on the use of a soluble receptor that consists of the extracellular domain of VEGFR1 and VEGFR2 fused to the Fc portion of human immunoglobulin G1 and is believed to be a competitive inhibitor by acting as a trap for VEGF, thereby disrupting the VEGF cascade and inhibiting the formation of new blood vessels in tumors. (Holash, et al., 2002, PNAS, vol. 98, no. 17, pp 11393-1398.).

In another use of soluble receptors, in inflammatory disease, and particularly in rheumatoid arthritis, tumor necrosis factor-alpha (“TNF-α”) activation is needed to produce inflammation. The initiation of TNF-α-mediated events requires the binding of TNF-α homotrimers to the extracellular domains of cell surface receptors. These cell surface receptors then form multimers, and signal transduction subsequently occurs through the receptors intracellular domains. Although natural, soluble forms of the TNF-α receptors have been shown to exist naturally and are capable of acting as competitive inhibitors for binding of TNF-α to cell surface receptors, they are not enough to block the level of activity seen in most inflammatory diseases. The anti-arthritis drug etanercept, known as Enbrel® (trademark of Amgen, Inc., Thousand Oaks, Calif., USA), is a fully human trimeric fusion protein consisting of the extracellular ligand-binding domain of the human TNF-α receptor linked to the Fc portion of human IgG1. It acts as a competitive inhibitor of the binding of TNF-α to cell surface TNF receptors and thereby inhibits TNF-α-induced inflammatory activity in the joints of rheumatoid arthritis patients. Etanercept acts as a cytokine carrier and TNF-α antagonist, rendering TNF-α biologically inactive. (Goffe, et al. 2003, J. Am. Acad. Dermatol., vol. 49, no. 2, pp. S105-S111; Goldenburg, M., 1999, Clinical Therapeutics, vol. 21, no. 1, pp. 75-87).

Another drug that also targets the TNF-α receptors is infliximab. This is a monoclonal chimeric antibody to TNF-α to prevent the binding that must occur, but it binds to both membrane-bound and soluble TNF-α, possibly causing some unwanted side effects.

Receptor tyrosine kinases are key to the growth of some breast cancers. In general, receptor tyrosine kinases are glycoproteins which consist of (1) an extracellular domain that is able to bind with a specific ligand, (2) a transmembrane region, (3) a juxtamembrane domain where the receptor may be regulation by, for instance, protein phosphorylation, (4) a tyrosine kinase domain that is the enzymatic component of the receptor, and (5) a carboxyterminal tail. (Davis, et al., U.S. Pat. No. 6,441,137, Aug. 27, 2002). For breast cancers, the erbB family of type I receptor tyrosine kinases are the most important ones to date. These include erbB1 (also known as HER1), erbB2 (HER2/neu), erbB3 (HER3), and erbB4 (HER4). These receptor tyrosine kinases are widely expressed in epithelial, mesenchymal, and neuronal tissues. Overexpression of erbB2 or erbB1 has been correlated with a poorer clinical outcome in some breast cancers and a variety of other malignancies.

In their inactive state, erbB receptors exist as monomers. Upon binding with a soluble ligand, conformational changes occur within the receptor which results in the formation of receptor homo- and heterodimers, i.e., the activated receptor form. Ligand binding and subsequent homo- or heterodimerization stimulates the catalytic activity of the receptor through autophosphorylation, that is, the individual monomers will phosphorylate each other on tyrosine residues. This results in further stimulation of receptor catalytic activity. In addition, some of the phosphorylated tyrosine residues provide a docking site for downstream signaling molecules.

Activation of erbB receptors results in different downstream events such as proliferation and cell survival. These different outcomes occur through different signaling pathways, which depend on the particular ligand which binds to a particular receptor. Ligand binding then dictates the composition of the homo- or heterodimers that form as a result. Nurmerous studies have now shown that the type of bound ligand, and subsequent type of homo- or heterodimer formed, results in the differential phosphorylation of tyrosine residues on the activated erbB receptors. As an example, the neuregulins (“NRGs”, also known as heregulins) are a family of ligands that bind to erbB receptors and elicit different responses including proliferation, differentiation, survival, and migration. NRG1β and NRG2β can bind to erbB3 and induce erbB2/erbB3 heterodimers, however, only NRG1β stimulates differentiation of breast cancer cells in culture. The reason for this is the recruitment of different downstream signaling molecules to the activated erbB2/erbB3 heterodimers when NRG1β is bound as compared to when NRG2β is bound. For example, although NRG1β and NRG2β result in similar overall levels of erbB2 tyrosine phosphorylation, only NRG1β resulted in the association of P13K (p85), SHP2, Grb2, and Shc with the receptor.

Another family of soluble peptide ligands regulates erbB receptor signaling, and includes epidermal growth factor (“EGF”) and transforming growth factor α (“TGF-α”), each of which binds to receptor erbB1. Increased expression of the ligands EGF or TGF-α has been reported as a poor prognostic indicator in some cancer patients, and locally increased concentrations of EGF or other ligands in the tumor microenvironment appear to be capable of maintaining heterodimers in an activated state even in the absence of receptor overexpression.

In breast cancers, erbB1 and erbB2 heterodimerize to activate survival and proliferation pathways. ErbB1, erbB2, and erbB4 all possess functional catalytic domains, which are a key property in activating these pathways. Several therapeutic strategies aimed at inhibiting the action of erbB1 and erbB2 are known. One of these is the use of antibodies to bind to the extracellular domain of these receptors and cause receptor degradation, and another is the use of small molecule inhibitors that bind to the active kinase domain on the intercellular domain of these receptors. Unfortunately, none of these therapies has been able to totally eradicate the growth of breast cancers.

One reason for this may be that not all of the erbB receptors have functional catalytic domains that can be inhibited by small molecules. The erbB3 receptor tyrosine kinase is unusual among receptor kinases in that its catalytic domain is defective. Although there are other known kinase defective receptors, i.e., CCK-4m VIK/RYK, K1g, and Ror1, these are orphan receptors without known ligands. ErbB3 differs from these other receptors because it can bind to ligands such as heregulin (known as HRG or neuregulin (NDF)). Even though it lacks catalytic activity, erbB3 is important in that it will heterodimerize with erbB2 and erbB4 to form erbB2/erbB3 and erbB3/erbB4 heterodimers, thereby stimulating signaling activity in breast cancer (Lee, et al., 2001, Cancer Research 61, pp 4467-4473).

Naturally occurring truncated erbB3 receptors, which consist only of the extracellular domain of the erbB3 receptor, have been identified. These natural, soluble receptors have been shown to bind ligand and therefore function as competitive inhibitors of cellular ErbB3 receptors. These naturally occurring soluble receptors do not exist in quantities large enough to offer a therapeutic effect to patients having breast cancer. In particular, Lee et al. discuss the discovery of a p85-soluble erbB3 receptor and its activity as a competitive inhibitor of erbB3 ligands. It was found that this receptor is a naturally occurring ErbB3 inhibitor in vitro, by preventing HRG from binding with cell surface receptors. It may be considered as one of many negative extracellular regulators. (Lee, et al., 2001, Cancer Research, 61: 4467-4473).

It is an objective of the invention to provide erbB3 ligand antagonists that competitively bind to erbB3 ligands, thereby reducing or eliminating the availability of such ligands for binding with cellular erbB3 receptors.

It is preferably an objective of the invention to provide methods of treating cancer by the administration of erbB3 ligand antagonists either as a single agent or in combination with other cancer therapies.

### SUMMARY OF THE INVENTION

A new ErbB-based ligand binding molecule is disclosed along with its method of preparation and use. The binding molecule can be a protein expressed from a recombinant DNA molecule. The protein can contain an ErbB extracellular domain that can bind to activating ligands. These binding elements can act as traps to bind and sequester circulating ligands, thus making them unavailable for binding to cellular ErbB receptors.

Preferably, the protein can include portions of ErbB receptors and will bind to ErbB ligands.

Additional features and advantages are described herein, and will be apparent from, the following Detailed Description and the figures.

### BRIEF DESCRIPTION OF THE DRAWINGS

FIG. 1: Schematic of the ErbB Single Trap mechanism of action.

FIG. 2: Western blot of the ErbB3 Single Trap and ErbB Double Trap polypeptides.

### DETAILED DESCRIPTION OF THE INVENTION

The present specification describes a binding molecule capable of binding ligands to a receptor, such as an ErbB receptor. The binding molecule is termed a “single trap” for purposes of this disclosure. In one embodiment, the molecule has substantial affinity for a subset of all ErbB ligands. The molecule can be used as a monotherapy or in combination therapies, for example with EGFR and ErbB2 inhibitors in those tumors that overexpress, or have alterations in, both the ErbB receptors and ligands.

In an embodiment the invention relates to a monovalent binding molecule having substantial binding affinity for those ligands that bind to a particular ErbB receptor. Generally, the ligands will be distinct ligands that bind to distinct receptors. It is preferred that the binding molecule be soluble in aqueous solutions. Alternatively, the binding molecule can be in a formulation that renders it functionally soluble or hydrophilic, such as a liposome formulation.

In an embodiment the binding molecule can be a soluble portion of an extracellular domain of a receptor. Any suitable receptor can be utilized in the binding molecule. Suitable receptors will generally contain extracellular domains that contain all of the determinants necessary and sufficient for ligand binding. These determinants can be located on a corresponding mRNA or genetic structure that can be isolated either directly from the genome or from the cDNA derived from its native host cells. In certain embodiments the family of ErbB receptors can be used to create binding molecules. To this end, the binding molecule can include extracellular ligand binding domains of ErbB receptors, for example ErbB1, ErbB3 or ErbB4. The binding domains can exist with other elements on the polypeptide chain so long as suitable binding activity for the receptor ligand is maintained.

The binding molecules can include amino acid sequences expressed from a recombinant DNA molecule. The recombinant DNA molecule can include a first nucleotide sequence encoding a portion of a receptor protein. Other elements can be included in the binding molecules, such as the Fc portion of IgG1. The use of the Fc portion of human IgG1 is a design well known to those skilled in the art of therapeutics, and is known to serve two purposes. The first is to allow the monomeric soluble receptors to oligomerize to higher order structures, such as dimers and trimers. The second purpose is to generate a more stable molecule resulting in a longer half-life in vivo than a molecule without the Fc portion of the IgG1 molecule. Another element can be a linker to operably connect the ligand binding element with the IgG1-Fc element. One example can be a glycine-serine (Gly4Ser)3 linker. Another element can be a protease recognition sequence to allow the IgG1-Fc element to be removed from the molecule if so desired. Exemplary sequences include Factor Xa or TEV protease recognition sequence.

In certain embodiments the recombinant DNA molecule includes a sequence for ErbB1. In certain embodiments the recombinant DNA molecule includes a sequence derived from ErbB3. In other embodiments the recombinant DNA molecule can include a sequence derived from ErbB4. In any of these cases, the chosen sequence will have a substantial capacity to bind their corresponding ErbB ligands.

In certain embodiments the receptor sequences are cloned into a recombinant DNA construct in an arrangement with transcription and translation sequences such that the binding molecule can be expressed in a suitable host. It is well within the skill of one having skill in the art to select transcription and translation sequences that can be used in suitable hosts. In many circumstances receptors are glycosylated and glycosylation can influence ligand binding. Thus, the selection of a host can depend on the glycosylation pattern generated by the host cell. For example, in the case of an ErbB-containing binding molecule a mammalian host cell can be used.

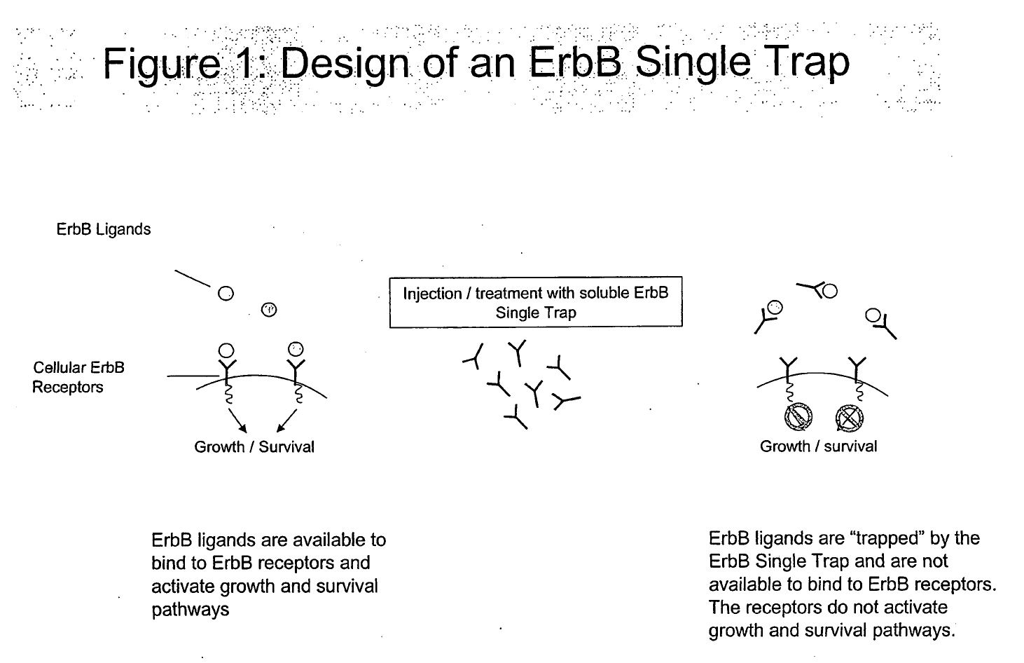

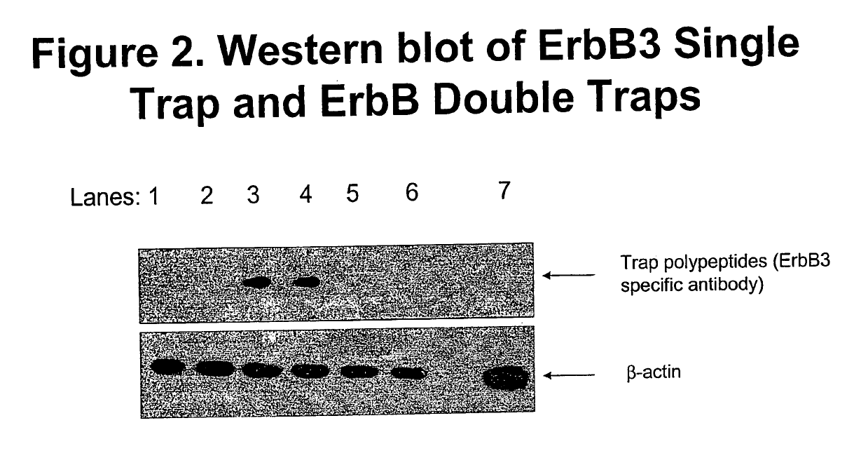

Exemplary embodiments of binding molecules are illustrated diagrammatically in FIG. 1. Detection of ErbB ligand binding domains is shown by recognition with extracellular domain specific antibodies to ErbB receptors by western blot (FIG. 2).

Methods for using pharmaceutical compositions of the inventive compounds are also provided. Such pharmaceutical compositions can be for administration for injection, or for oral, pulmonary, nasal, transdermal or other forms of administration. In general, the invention encompasses pharmaceutical compositions comprising effective amounts of a binding molecule of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., TWEED™ 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder, such as lyophilized form. Implantable sustained release formulations are also contemplated, as are transdermal formulations.

Pharmaceutically acceptable carriers include carbohydrates such as trehalose, mannitol, xylitol, sucrose, lactose, and sorbitol. Other ingredients for use in formulations may include DPPC, DOPE, DSPC and DOPC. Natural or synthetic surfactants may be used. PEG may be used (even apart from its use in derivatizing the protein or analog). Dextrans, such as cyclodextran, may be used. Bile salts and other related enhancers may be used. Cellulose and cellulose derivatives may be used. Amino acids may be used, such as use in a buffer formulation.

Also, the use of liposomes, microcapsules or microspheres, inclusion complexes, or other types of carriers is contemplated.

The dosage regimen involved in a method for treatment will be determined by the attending physician, considering various factors which modify the action of drugs, e.g. the age, condition, body weight, sex and diet of the patient, the severity of disease, time of administration and other clinical factors. Generally, the daily regimen should be in the range of 0.1-1000 micrograms of the inventive compound per kilogram of body weight, preferably 0.1-150 micrograms per kilogram.

### EXAMPLE 1

The present example demonstrates the construction of one representative composition of a single trap molecule having an ErbB receptor extracellular domain, an ErbB3 extracellular domain in a single recombinant genetic construct.

The ErbB single trap can be designed to bind to different ligands of the ErbB family by incorporating the extracellular domains of either ErbB1, ErbB3 or ErbB4.

A truncated form of ErbB3 was cloned into the pEF-IRES-P plasmid by Dr. Yosef Yarden's laboratory at the Weizmann Institute in Rehovot, Israel. This construct consists of the first three extracellular domains of ErbB3 called LI (domain I), SI (domain II), LII (domain III) and a portion of SII (domain IV). Other protein domains fused to the 3′ end of this fragment include a PKA phosphorylation site, a Factor Xa cleavage site, and the Fc fragment of human IgG1 attached to the 3′ end. This entire fragment was then cloned into the NheI-NotI sites of the pEF-IRES-P plasmid to yield the pEF-ECD3IgG-IRES-P plasmid.

pEF-ECD3IgG-IRES-P plasmid was altered to generate the pEF-ECD3-IRES-P plasmid. The ECD3IgG was truncated such that only the LI, SI, LII and a portion of SII domains of ErbB3 were expressed. Because there were no convenient restriction enzymes to accomplish this, PCR primers were designed to amplify a portion of the ErbB3 extracellular domain. These primers incorporated an Xho I site on the forward primer, as this was the nearest unique restriction enzyme site to the 3′ end of domain SII. The reverse primer incorporated a Not I site, as this was the original cloning site on the 3′ end of the construct, a nested Nhe I site and a Stop codon that overlapped the Nhe I site. The Nhe I site was incorporated inside the Not I site on the reverse primer to enable the construction of a pEF-IRES-P empty control plasmid because there was a Nhe I site at the 5′ end of the multiple cloning site (“MCS”) which would allow the entire ErbB3 extracellular domain fragment to be excised following construction of the ErbB3 Single Trap.

pEF-ECD3IgG-IRES-P was used as a template for a PCR reactions as follows: 25 ng of pEF-ECD3IgG-IRES-P plasmid, 2.5 μl NEB 10× Vent polymerase buffer, 0.5 μl dNTP (10 mM solution each of dATP, dCTP, dGTP and dTTP), 0.5 μl forward and reverse primers and 0.5 μl of Vent polymerase (5000 units/mL). The sequences of the primers were: s-erbB3-XhoI, 5′ AGC TCT CGA GCA ACA TTG ATG GAT TTG TGA ACT GC (SEQ ID NO 1) and s-erbB3-NheI-NotI, 5′ AGC TGC GGC CGC TAG CTC AAC CAG GGC CTG GGC CCC AGC ATC (SEQ ID NO 2). PCR conditions were as follows: 95° C. for 2 min, followed by 22 cycles of 95° C. for 45 s, 55° C. for 45 s and 72° C. for 2 min, followed by a final extension at 72° C. for 5 min.

The amplified PCR fragment was electrophoresed on a 1% agarose gel and purified using a Qiagen Gel Extraction Kit. The PCR product and the pEF-ECD3IgG-IRES-P plasmid were digested overnight with Xho I and Not I, separated on a 1% agarose gel and purified using a Qiagen Gel Extraction kit. The Xho I-Not I PCR fragment was ligated into the Xho I-Not I digested pEF-ECD3IgG-IRES-P plasmid and transformed into DH5□ competent cells. Clones were screened for recombination by digestion with Xho I and Not I. This new construct, the pEF-ECD3-IRES-P plasmid, was then digested with Nhe I. The fragments were separated on a 1% agarose gel, purified using a Qiagen Gel Extraction Kit, and the pIRES backbone plasmid was religated to itself. This generated the pEF-IRES-P control plasmid, which did not possess any ErbB3 or IgG1-Fc sequences.

### EXAMPLE 2

This example demonstrates expression of a double trap molecule from a recombinant DNA molecule in a mammalian host cell and its purification in active form. The pEF-ECD3IgG-IRES-P plasmid, pEF-ECD3-IRES-P plasmid and pEF-IRES-P control plasmid were separately transfected into 293T cells, which were then selected on puromycin in order to generate a population of cells with stable integration of the plasmid. These transduced cells secrete the ECD3IgG and ECD3 Single Trap polypeptides into the culture medium.

The results of the western blot from the transfected 293T cells (FIG. 2) demonstrated that the ErbB3 Single Trap was not recognized by an ErbB3 specific antibody (lane 1), however polypeptides that express the soluble ErbB3 and soluble ErbB1 from the same polypeptide were detected by the antibody (lanes 3 and 4). These polypeptides are called ErbB Double Traps because they contain extracellular ligand binding portions of 2 different ErbB extracellular ligand binding domains (Bob: should we cite our other patent here?). The only difference between the ErbB3 extracellular ligand binding portion used for the Single Trap and the Double Trap was an addition of 3 amino acids at the carboxy terminus of the ErbB3 extracellular ligand binding portion in the Double Trap. Therefore, the ErbB3 single trap will be modified to include these 3 additional amino acids, as well as the TEV protease recognition sequence and histidine tag which comprise the double trap polypeptides but which is not present in the current form of the ErbB3 Single Trap. These elements can be added to the ErbB3 Single Trap polypeptide by known methods such as PCR.

### EXAMPLE 3

To test the functionality of the ErbB3 Single Traps, conditioned medium from the 293T cells was collected, filtered and used to culture BT474 breast cancer cells. A significant reduction in cell number was observed after 48 hrs in the BT474 cells cultured with medium from 293T cells that express the pEF-ECD3-IRES-P plasmid compared with cells that were cultured with conditioned medium from 293T cells that express the pEF-IRES-P control vector.

The invention is claimed as follows:

1. A binding molecule having binding affinity for an ErbB ligand, wherein the entire binding molecule is the extracellular region of an ErbB receptor or portion thereof.

2. The binding molecule of claim 1, wherein the binding molecule is substantially soluble in aqueous solution.

3. The binding molecule of claim 1, wherein the binding molecule further comprises an extracellular domain of an ErbB receptor.

4. The binding molecule of claim 1, further comprising an extracellular domain from ErbB1.

5. The binding molecule of claim 1, further comprising an extracellular domain from ErbB3.

6. The binding molecule of claim 1, further comprising an extracellular domain from ErbB4.

7. A eukaryotic cell comprising a recombinant DNA molecule comprising a nucleotide sequence encoding a portion of an ErbB receptor protein.

8. The eukaryotic cell of claim 7, wherein the cell produces a binding molecule having binding affinity for an ErbB ligand or ligands.

9. The eukaryotic cell of claim 7, wherein the cell produces a binding molecule having binding affinity for an ErbB ligand wherein the binding molecule is soluble in an aqueous solution.

10. The eukaryotic cell of claim 7, wherein the binding molecule is transported to the exterior of the cell and into the surrounding media.

11. The eukaryotic cell of claim 7, wherein the binding molecule further comprises an extracellular domain of an ErbB receptor.

12. The eukaryotic cell of claim 7, further comprising an extracellular domain from ErbB1.

13. The eukaryotic cell of claim 7, further comprising an extracellular domain from ErbB3.

14. The eukaryotic cell of claim 7, further comprising an extracellular domain from ErbB4.

15. The eukaryotic cell of claim 7, wherein the recombinant DNA molecules contains the sequences of a Factor Xa cleavage site, and an Fc portion of human immunoglobulin G1.

16. The eukaryotic cell of claim 15, wherein the cell produces a binding molecule having binding affinity for an ErbB ligand or ligands.

17. The eukaryotic cell of claim 15, wherein the cell produces a binding molecule having binding affinity for an ErbB ligand wherein the binding molecule is soluble in an aqueous solution.

18. The eukaryotic cell of claim 15, wherein the binding molecule is transported to the exterior of the cell and into the surrounding media.

19. The eukaryotic cell of claim 15, wherein the binding molecule further comprises an extracellular domain of an ErbB receptor.

20. The eukaryotic cell of claim 15, further comprising an extracellular domain from ErbB1.

21. The eukaryotic cell of claim 15, further comprising an extracellular domain from ErbB3.

22. The eukaryotic cell of claim 15, further comprising an extracellular domain from ErbB4.
